(19) United States
(12) Patent Application Publication
(10) Pub. No.: US 2025/0383976 A1
(43) Pub. Date: Dec. 18, 2025

(54) System and Method for Automated Testing of Configuration Data

(71) Applicant: Cognizant Technology Solutions US Corp, Teaneck, NJ (US)

(72) Inventors: Thomas Russell Newman, Littleton, CO (US); Amanda Jean Dussault, Wake Forest, NC (US); Lisa Kay Warfield, Hampton, VA (US); Nicholas Patrick Hunter, Denver, CO (US)

(21) Appl. No.: 19/237,413

(22) Filed: Jun. 13, 2025

Related U.S. Application Data
(60) Provisional application No. 63/659,866, filed on Jun. 14, 2024.

(51) Int. Cl. G06F 11/3668; G06F 40/205; G16H 10/60

(52) U.S. Cl. CPC G06F 11/3688; G06F 11/3684; G06F 40/205; G16H 10/60

(57) ABSTRACT

A system and method for automated testing of configuration data is provided. A first input type and a second input type related to healthcare claims data are captured by executing a Gen AI model. A configuration data document analysis model is executed for determining relationships between the different type of healthcare claims data associated with the first input type and identifying one or more characteristics associated with the first input type. Synthetic healthcare claims datasets are generated based on the determined relationships and the identified characteristics associated with the first input type and the second input type. A test case is generated by carrying out search and cloning of one or more healthcare plan parameters associated with a test case plan. The generated test case is executed based on one or more test scope elements for testing the configuration data and determining accuracy of configuration data.

### CROSS-REFERENCE TO RELATED APPLICATIONS

This application claims the benefit of U.S. patent application No. 63/659,866 filed on Jun. 14, 2024, the disclosure of which is incorporated herein by reference in its entirety.

### FIELD OF THE INVENTION

The present invention relates generally to the field of configuration data testing. More particularly, the present invention relates to a system and a method for automated testing of configuration data associated with healthcare claims data.

### BACKGROUND OF THE INVENTION

Large amounts of datasets are generated for catering to requirements of users working in a particular domain, and processing and testing of such datasets are generally carried out using different software applications. One such domain is healthcare, for which the software applications are developed for processing healthcare plans data. Typically, healthcare plans data requires processing of healthcare claims, which is done by using software applications configured with different types of configuration data (e.g., a benefit package data, terms of a negotiated provider contract, etc.). The configuration data is analyzed, tested and validated for the healthcare plan. Conventionally, the configuration data is developed and thereafter validated within specific software application functionality and in installations known as non-production environments. It has been observed that the effort required to validate all the configuration data which are necessary for the healthcare plan is not only enormous and expensive but are also prone to inaccuracy and may not adequately cover all the necessary configured scenarios.

Further, typically, the most common approach for Quality Assurance (QA) testing requires generation of one or more non-production environments in which complex sets of software applications, which are developed to emulate real production installations, are deployed. The non-production environments are thereafter loaded with one or more copies of actual operational data (i.e., production data). Further, QA testing of the configuration data employs production data to execute testing methods to verify accuracy and consistency of the configuration data. It has been observed that the use of production data is associated with a substantial privacy risk. Healthcare plan data is prone to cyber-attacks as the production data, that includes Personal Health Information (PHI) of a patient, may be replicated and is at a greater risk of being exposed or copied. Also, one or more existing data protection techniques, which may be applied for protecting healthcare plans data, provide ineffective data protection.

In light of the aforementioned drawbacks, there is a need for a system and a method which provides for an automated and efficient testing of configuration data. There is a need for a system and a method which effectively addresses the concerns of data protection and privacy associated with data used in configuration testing. Further, there is a need for a system and a method for carrying out cost-effective and time efficient QA testing of configuration data.

### SUMMARY OF THE INVENTION

In various embodiments of the present invention, a system for automated testing of configuration data is provided. The system comprises a memory storing program instructions, a processor executing instructions stored in the memory, and a data testing engine executed by the processor. The data testing engine captures a first input type and a second input type related to healthcare claims data via a Graphical User Interface (GUI) by executing a Generative Artificial Intelligence (Gen AI) model. The data testing engine parses the first input type and the second input type for extracting healthcare plans data in a segmented format. A configuration data document analysis model is executed for determining relationships between different types of the healthcare claims data associated with the first input type and identifying one or more characteristics associated with the first input type. The data testing engine generates synthetic healthcare claims datasets based on the determined relationships and identified characteristics associated with the first input type and the second input type. The data testing engine generates a test case by carrying out search and cloning of one or more healthcare plan parameters associated with a test case plan. The synthetic healthcare claims datasets are embedded into the generated test case. The data testing engine executes the generated test case based on one or more test scope elements for testing the configuration data and determining accuracy of the configuration data.

In various embodiments of the present invention, a method for automated testing of configuration data is provided. The method is implemented by a processor executing instructions stored in a memory. The method comprises capturing a first input type and a second input type related to healthcare claims data via a GUI by executing a Gen AI model. The method comprises parsing the first input type and the second input type for extracting healthcare plans data in a segmented format. A configuration data document analysis model is executed for determining one or more relationships between different types of the healthcare claims data associated with the first input type and identifying one or more characteristics associated with the first input type. The method comprises generating synthetic healthcare claims datasets based on the determined relationships and identified characteristics associated with the first input type and the second input type. The method comprises generating a test case by carrying out search and cloning of one or more healthcare plan parameters associated with a test case plan. The synthetic healthcare claims datasets are embedded into the generated test case. The method comprises executing the generated test case based on one or more test scope elements for testing the configuration data and determining accuracy of the configuration data.

In various embodiments of the present invention, a computer program product is provided. The computer program product comprises a non-transitory computer-readable medium having computer program code stored thereon, the computer-readable program code comprising instructions that, when executed by a processor, causes the processor to capture a first input type and a second input type related to healthcare claims data via a GUI by executing a Gen AI model. The first input type and the second input type are parsed for extracting healthcare plans data in a segmented format. A configuration data document analysis model is executed for determining relationships between different types of the healthcare claims data associated with the first input type and identifying one or more characteristics associated with the first input type. Synthetic healthcare claims datasets are generated based on the determined relationships and the identified characteristics associated with the first input type and the second input type. A test case is generated by carrying out search and cloning of one or more healthcare plan parameters associated with a test case plan. The synthetic healthcare claims datasets are embedded into the generated test case. The generated test case is executed based on one or more test scope elements for testing the configuration data and determining accuracy of configuration data.

### BRIEF DESCRIPTION OF THE ACCOMPANYING DRAWINGS

The present invention is described by way of embodiments illustrated in the accompanying drawings wherein:

FIG. 1 is a detailed block diagram of a system for automated testing of configuration data, in accordance with an embodiment of the present invention;

FIG. 2 illustrates a flowchart depicting an extraction and formatting process of a first input type, in accordance with an embodiment of the present invention;

FIGS. 3A-3N illustrate screenshots of a Graphical User Interface (GUI) depicting test case data extraction process for use in test case execution, in accordance with an embodiment of the present invention;

FIG. 4 illustrates a flowchart depicting a validation process of the configuration data, in accordance with an embodiment of the present invention;

FIGS. 5 and 5A flowchart depicting a method for automated testing of configuration data, in accordance with an embodiment of the present invention; and

FIG. 6 illustrates an exemplary computer system in which various embodiments of the present invention may be implemented.

### DETAILED DESCRIPTION OF THE INVENTION

The disclosure is provided in order to enable a person having ordinary skill in the art to practice the invention. Exemplary are embodiments herein provided only for illustrative purposes and various modifications will be readily apparent to persons skilled in the art. The general principles defined herein may be applied to other embodiments and applications without departing from the scope of the invention. The terminology and phraseology used herein is for the purpose of describing exemplary embodiments and should not be considered limiting. Thus, the present invention is to be accorded the widest scope encompassing numerous alternatives, modifications, and equivalents consistent with the principles and features disclosed herein. For purposes of clarity, details relating to technical material that is known in the technical fields related to the invention have been briefly described or omitted so as not to unnecessarily obscure the present invention.

The present invention would now be discussed in context of embodiments as illustrated in the accompanying drawings.

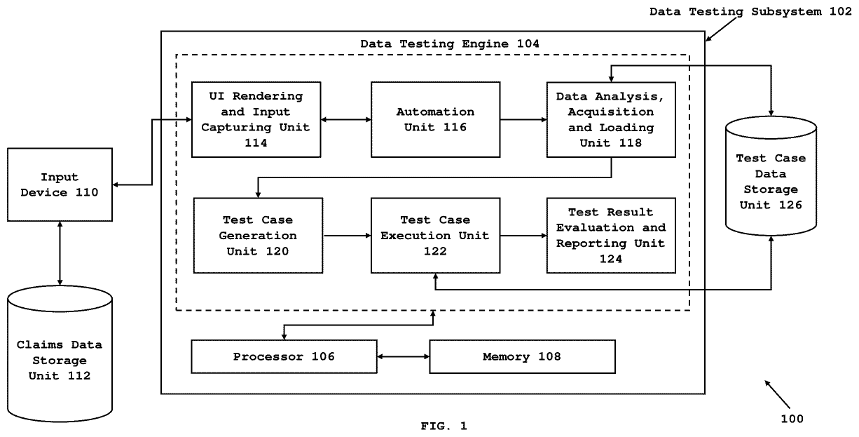

FIG. 1 is a detailed block diagram of a system 100 for automated testing of configuration data, in accordance with various embodiments of the present invention. Referring to FIG. 1, in an embodiment of the present invention, the system 100 comprises a data testing subsystem 102, an input device 110, a claims data storage unit 112, and a test case data storage unit 126. In an exemplary embodiment of the present invention, the input device 110, the claims data storage unit 112, and the test case data storage unit 126 are connected to the subsystem 102 via a communication channel (not shown). The communication channel (not shown) may include, but is not limited to, a physical transmission medium, such as, a wire, or a logical connection over a multiplexed medium, such as, radio a channel in telecommunications and computer networking. Examples of radio channel in telecommunications and computer networking may include, but are not limited to, a local area network (LAN), a metropolitan area network (MAN) and a wide area network (WAN).

In an embodiment of the present invention, the subsystem 102 is configured with a built-in mechanism to carry out Quality Assurance (QA) testing of configuration data related to healthcare claims in an automated manner. The subsystem 102 is configured to implement artificial intelligence (AI), machine learning (ML) techniques and generative AI (Gen AI) techniques for carrying out testing of configuration data associated with healthcare claims processing. In an exemplary embodiment of the present invention, the Gen AI technique guides the user (e.g., a QA tester) through the QA testing process based on processing of various approaches and requirements for QA testing provided as prompts by the user, and providing output based on the prompt for carrying out testing of the configuration data associated with healthcare claims processing. Further, the subsystem 102 is configured to generate and render an intelligent Graphical User Interface (GUI) for carrying out different functionalities associated with QA testing of configuration data associated with healthcare claims processing.

In an embodiment of the present invention, the subsystem 102 comprises a data testing engine 104 (engine 104), a processor 106, and a memory 108. In various embodiments of the present invention, the engine 104 has multiple units which work in conjunction with each other for carrying out Quality Assurance (QA) testing of configuration data related to healthcare claims in an automated manner. The various units of the engine 104 are operated via the processor 106 specifically programmed to execute instructions stored in the memory 108 for executing respective functionalities of the units of the engine 104 in accordance with various embodiments of the present invention.

In another embodiment of the present invention, the subsystem 102 may be implemented in a cloud computing architecture in which data, applications, services, and other resources are stored and delivered through shared datacenters. In an exemplary embodiment of the present invention, the functionalities of the subsystem 102 are delivered to a user as Software as a Service (Saas) and/or a Platform as a Service (Paas) over a communication network.

In another embodiment of the present invention, the subsystem 102 may be implemented as a client-server architecture. In this embodiment of the present invention, a client terminal accesses a server hosting the subsystem 102 over a communication network. The client terminals may include but are not limited to a smart phone, a computer, a tablet, microcomputer or any other wired or wireless terminal. The server may be a centralized or a decentralized server.

In an embodiment of the present invention, the engine 104 comprises a User Interface (UI) rendering and input capturing unit 114, an automation unit 116, a data analysis, acquisition and loading unit 118, a test case generation unit 120, a test case execution unit 122, and a test result evaluation and reporting unit 124.

In operation, in an embodiment of the present invention, the User Interface (UI) rendering and input capturing unit 114 is configured to render a Graphical User Interface (GUI) on the input device 110 for providing dashboards in order to capture one or more inputs related to healthcare claims data. The input device 110 is an electronic device. In an exemplary embodiment of the present invention, the input device 110 may include, but is not limited to, a computer, a laptop, a tablet, and a smartphone. The GUI is developed using a distributed development and consolidated delivery approach. Further, the GUI is embedded with various web components for carrying out various functionalities of capturing healthcare claims data. In an embodiment of the present invention, the dashboard is configured to capture a first input type and a second input type related to healthcare plan data. The first input type relates to healthcare claims data which may include, but is not limited to, healthcare claims type (e.g., healthcare insurance plan), Evidence Of Healthcare Plan Coverage (EOC), healthcare benefit summaries, healthcare plan contract terms, documents related to healthcare plan, health issue coverage data related to healthcare plan, patient personal data, patient health issue data, previous patient data (e.g., prior year's health care claim may have been “cloned” and used to create basis for the new healthcare claims), healthcare claims data storage location. The first input type may be provided in a pre-defined manner including manually, by uploading directly or by fetching from the claims data storage unit 112 integrated with the input device 110. The second input type relates to healthcare claims testing data, which may include, but is not limited to, types of testing to be performed, and location of testing. The type of testing may include, but is not limited to, validation testing and regression or parallel testing.

In an embodiment of the present invention, the automation unit 116 is configured to receive the first input type and the second input type. In an embodiment of the present invention, the automation unit 116 is configured with built-in Gen AI model, and pre-generated AI and ML models for processing the healthcare claims data based on the received first input type and the second input type. In an embodiment of the present invention, the automation unit 116 in communication with the UI rendering and input capturing unit 114 is configured to execute a Gen AI model such as Open AIR, Gemini®, Azure® open AI, etc., to guide the user through the GUI for capturing the first input type and the second input type in an efficient manner. For example, the user may provide a prompt related to the QA testing of the configuration data for a healthcare plan. The configuration data relates to parameters for determining how insurance claims are processed, adjudicated, and paid. The prompt may include, but is not limited to, determining where the configuration data was generated, guidance on how the configuration should be tested, determining whether prior results exist, determining if similar configuration exists, determining scope of QA testing and what type of results are to be expected. In an embodiment of the present invention, a semantic similarity analysis is carried out by the automation unit 116 for determining meaning of the prompt provided by the user. The automation unit 116 is configured to convert the words provided in prompts to vectors. The words are converted to vectors by implementing techniques such as, but are not limited to, a Global Vectors for Word Embeddings (GloVe) technique, auto-tokenizers, etc. Further, the vectors associated with semantically related words are clustered by the automation unit 116 by determining proximity of the vectors. The automation unit is 116 configured to implement a K-means clustering technique for clustering the vectors associated with semantically related words. The clustering of semantically related words aids in efficient natural language processing in a dynamic and intelligent manner. In an embodiment of the present invention, the automation unit 116 implementing the Gen AI model provides a required output to the user for suitably capturing the first input type and the second input type. In various embodiments of the present invention, the automation unit 116 creates an Artificial Intelligence (AI) based dictionary comprising of association of phrases, words and similies which is continuously updated and refined and carries out probabilistic prediction 41 the phrases, words and similies, thereby enhancing the AI-based dictionary's ability to mirror natural language use. Each time, for instance, healthcare plans data is processed, the automation unit 116 communicates to and provides for a database of word associations in the form of the AI-based dictionary. In an exemplary embodiment of the present invention, the database is implemented by employing a Retrieval-Augmented Generation (RAG) technique. The AI-based dictionary is dynamic and intelligent which is capable of evolving with language and providing valuable insights for various applications, from natural language processing to content recommendation systems, and bridging the gap between human language and machine understanding. The AI-based dictionary is implemented by employing RAG technique.

In an embodiment of the present invention, the automation unit 116 is configured to parse the received first input type and the second input type for extracting healthcare plans data in a segmented format. In an embodiment of the present invention, the automation unit 116, subsequent to parsing, executes a configuration data document analysis model for interpreting the first input type based on evaluating and identifying one or more relationships between the different type of healthcare claims data associated with the first input type. The interpretation of the first input type is carried out by identifying one or more characteristics associated with the first input type and the second input type. The one or more characteristics include, but are not limited to, determining type of health issue associated with a particular patient (e.g., broken arm is common with people of 12 years of age, if during a dermatologist visit a spot is noted, then it might be melanoma, etc.), explanation of healthcare claims coverage, healthcare claims benefit summaries, healthcare claims configuration document types, determining claims testing scope (e.g., determining claims testing needs such as positive/negative testing, which applications of functions need to be included, etc.), determining form of claims test result confirmation, etc.

In an embodiment of the present invention, the automation unit 116 performs a mapping operation between the first input type and the configuration data associated with the first input type based on the interpretation. For example, if the first input type relates to a claims benefit summary and is presented in a natural language form, then the first input type is mapped to the configuration data relating to Current Procedural Terminology (CPT) codes that are encoded in the configuration of a particular healthcare claims processing tool. In an embodiment of the present invention, the automation unit 116 generates one or more synthetic healthcare claims datasets associated with healthcare plan by executing a regression or classification machine learning model. The synthetic dataset claims are used for processing the healthcare claims. In an embodiment of the present invention, the automation unit 116 generates the synthetic healthcare claims datasets based on the identified relationships between the different type of first input type, the characteristics associated with the first input type and the second input type. In an example, for a particular healthcare claim, the “best, less common, or rare” characteristics for patient age, patient gender, physician type, facility type, cost share, etc., may be identified. The synthetic claims datasets are similar to actual Personal Health Information (PHI) of a patient. Advantageously, by employing synthetic claims datasets, privacy concerns associated with the usage of PHI are eliminated. The generated synthetic claims datasets also include information on a type of testing which is required to be carried out for the healthcare claims data. In an exemplary embodiment of the present invention, the type of testing includes regression or parallel testing and validation testing.

In an embodiment of the present invention, the automation unit 116 is configured to communicate with the data analysis, acquisition and loading unit 118 for processing and transmitting the generated synthetic claims datasets to the test case generation unit 120. The data analysis, acquisition and loading unit 118 is configured to analyze and process the synthetic claims datasets for generating healthcare claims models. In an embodiment of the present invention, the data analysis, acquisition and loading unit 118 implements large dataset analysis and processing techniques along with LLM management techniques for analyzing and processing the synthetic claims datasets. The large dataset analysis and processing techniques include, but are not limited to, Python® libraries like NumPy®, Pandas®, Shap® and Sklearn®. The LLM management techniques include, but are not limited to, Azure® ML and SQL Server. In an embodiment of the present invention, the data analysis, acquisition and loading unit 118 executes a configuration data analysis technique to generate the healthcare claim models.

In an embodiment of the present invention, the data analysis, acquisition and loading unit 118 is configured to evaluate one or more components associated with the synthetic claims datasets for healthcare claims processing and determine a suitable methodology for healthcare claims data processing. In an embodiment of the present invention, the data analysis, acquisition and loading unit 118 is further configured to include the prompts related to user interactions to validate the analysis process and identified characteristics with the first input type. For example, the prompts relate to best source for test case, scope of healthcare claims testing, mappings carried out, and one or more functional elements to be included in the build of the test case.

In an embodiment of the present invention, subsequent to processing of the synthetic claims datasets, the data analysis, acquisition and loading unit 118 is configured to extract and process the first input type fetched from the claims data storage unit 112 based on a pre-defined set of rules. The pre-defined set of rules executes an Application Programming Interface (API) for extracting a first set of rules from the claims data storage unit 112. The fetched first input type is loaded in a high structure SQL Server database (not shown) based on a set of pre-defined configurations. The extraction process creates an event bus message using a queuing protocol (e.g., a Rabbit MQ®) which fetches the first input type data from the SQL Server database (not shown) or from one or more third-party sources and converts the first input type into a pre-determined standard model including a NoSQL database schema standard (e.g., Fast Healthcare Interoperability Resources (FHIR) R5 standard). The pre-defined standard model is saved into a low structure database (e.g., MongoDB®) (not shown) with a unique data set ID. The pre-defined standard model is referred to as a data puddle which is in a pre-defined file format (e.g., as FHIR JSON). The data puddle is a discrete set of data. In an embodiment of the present invention, the first input type is masked prior to converting into the pre-defined standard model by executing a set of data alteration rules with respect to a database schema.

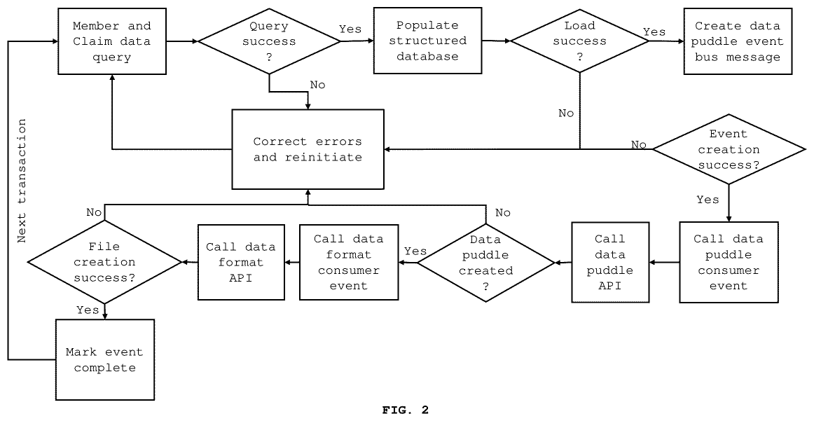

In an embodiment of the present invention, the pre-defined standard model stored in the low structure database is further retrieved and an event is triggered for formatting the pre-defined standard model. In an exemplary embodiment of the present invention, a data formatting application (e.g., EDITran) is executed for formatting the pre-defined standard model. After completion of the formatting event, a data file in a pre-defined file format is generated. For example, an X12 file, a JSON, a flat file is generated in a specified output folder. FIG. 2 illustrates a flowchart of the extraction and formatting process of the first input type. In an embodiment of the present invention, the formatted pre-defined standard model is transmitted to the test case data storage unit 126 which is a target database using a batch process upload technique for storage, re-use and retrieval of the test case data. In an embodiment of the present invention, a backup of the data puddles is created by using a database tool to create a full backup of the collections and a purging process is executed weekly for carrying out scans through the collection of data puddles older than the number of days set as retention days in a configuration file.

In an embodiment of the present invention, subsequent to completion of the data puddle event, the test case generation unit 120 is configured to generate a test case cycle. Firstly, the test case generation unit 120 determines one or more healthcare plan parameters associated with a test case plan. The one or more healthcare plan parameters, include, but are not limited to, determining specific service and diagnosis codes to match the requirements and descriptions of the configuration, healthcare claims, rules, billing, enrollment, contracts, authorizations, and identifying supporting particulars relating to implementing various encoded configuration data. Further, the test case generation unit 120 generates a test case by carrying out search and cloning of the one or more healthcare plan parameters associated with the test case plan. In an embodiment of the present invention, a configuration data analysis technique and a data cloning and PHI obfuscation are employed for generating the test case. In an example, PHI data may be used for testing. By obfuscating the PHI data for generating the test case it becomes difficult to find the obfuscated PHI data and, therefore, it is not possible to unmask the obfuscated PHI data, thereby providing security. In an embodiment of the present invention, the one or more healthcare plan parameters are synthetically generated by the test case generation unit 120 by using synthetic data creation technique. In an embodiment of the present invention, test case generation also includes generating data relating to proper execution of the test case, such as authorization, referrals, or care components. Further, the test case generation unit 120 fetches the synthetic healthcare claims datasets for embedding with the generated test case.

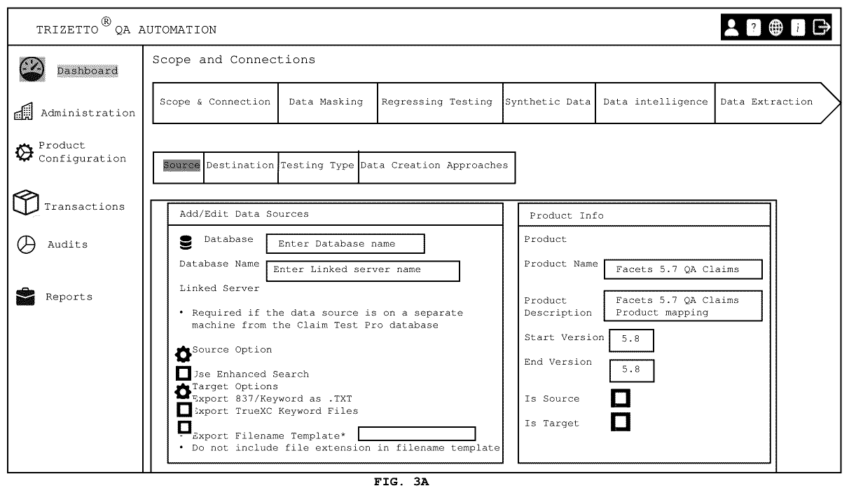

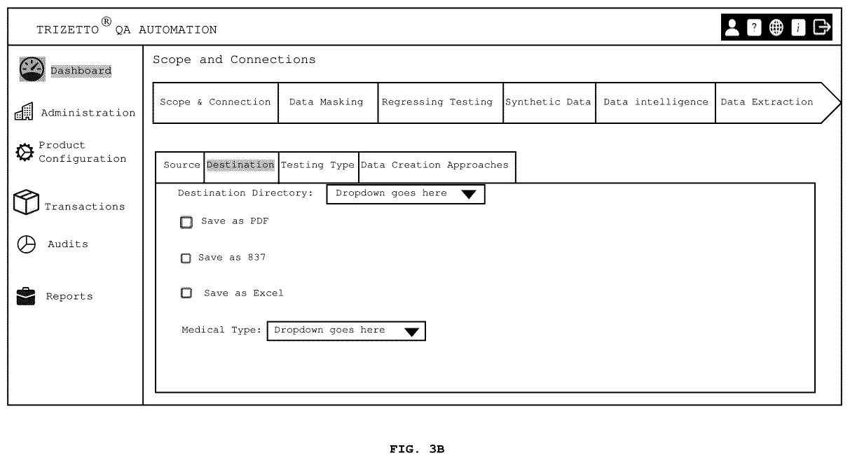

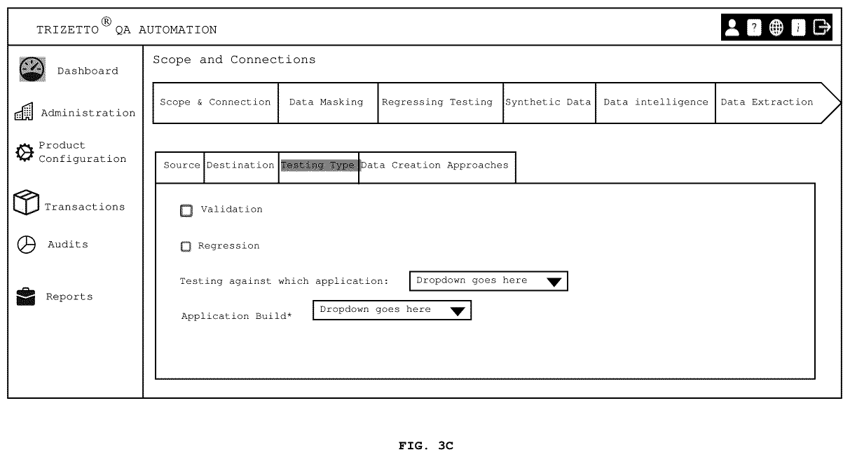

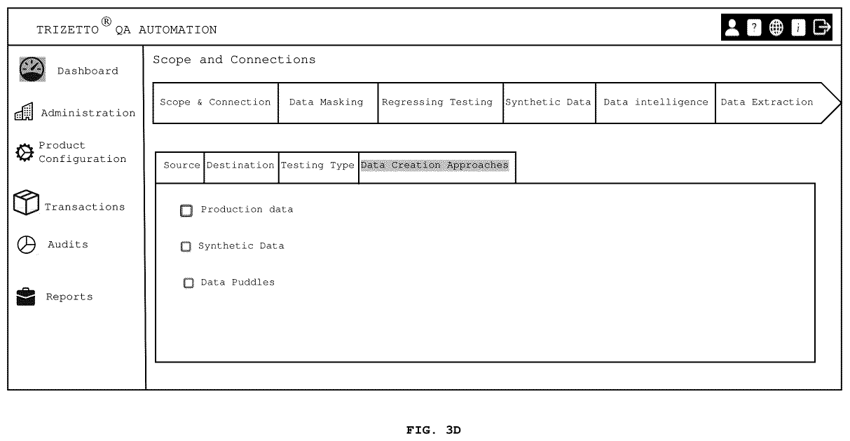

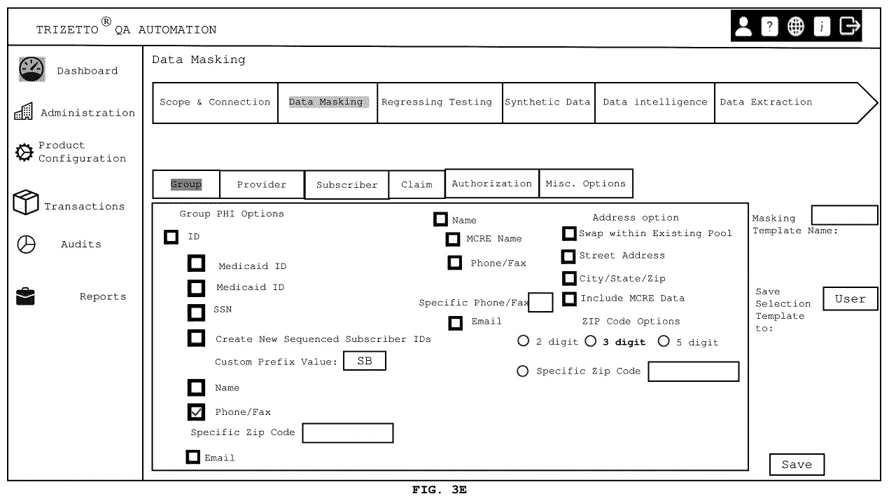

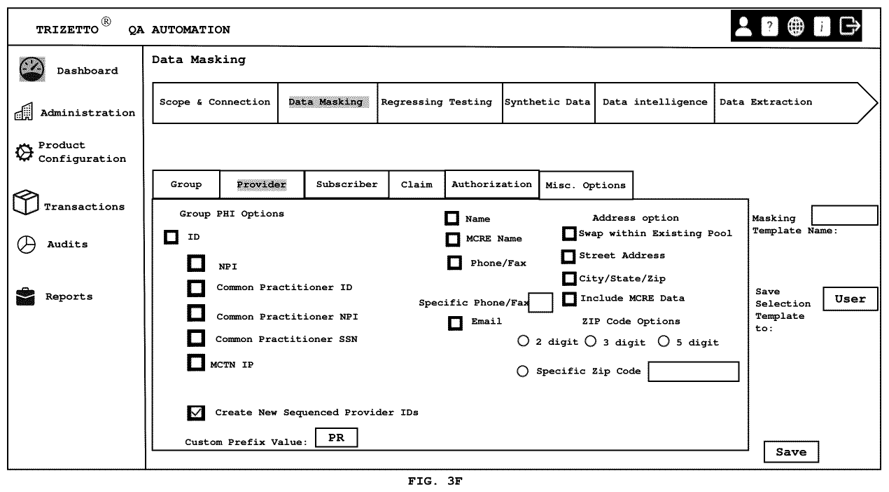

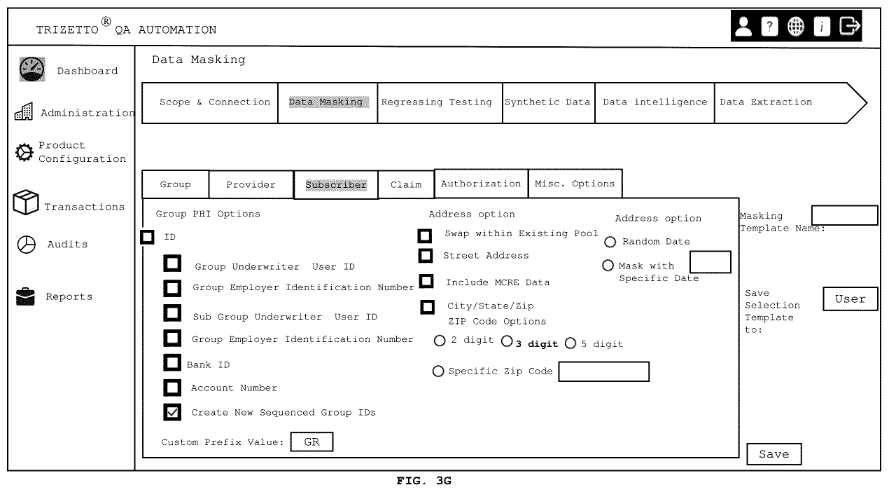

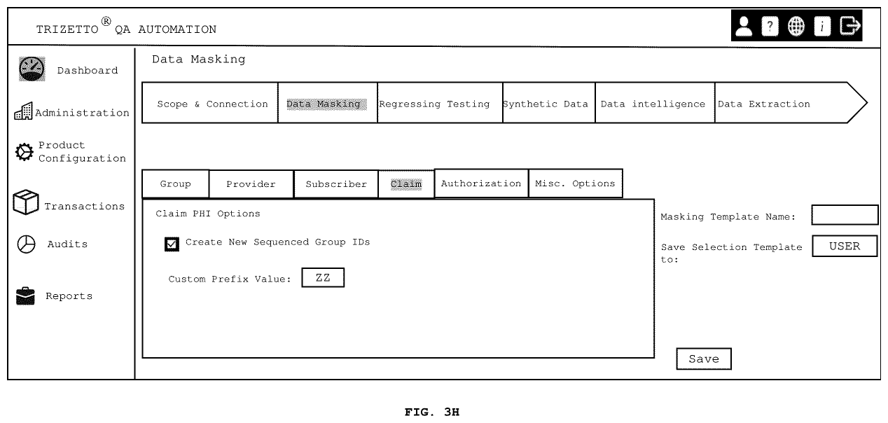

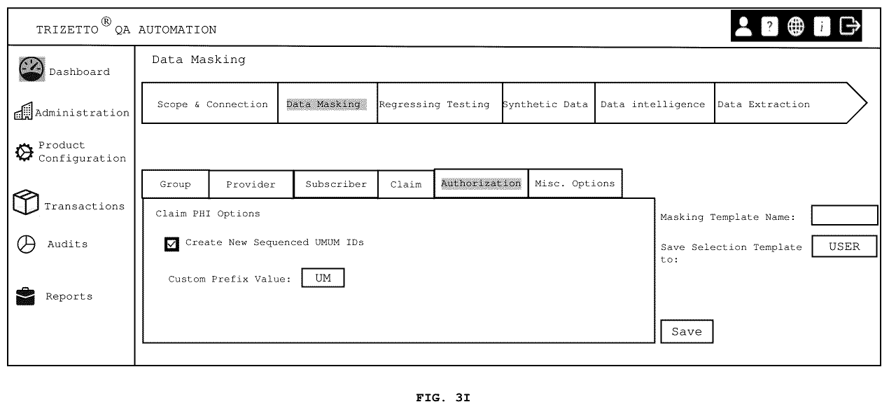

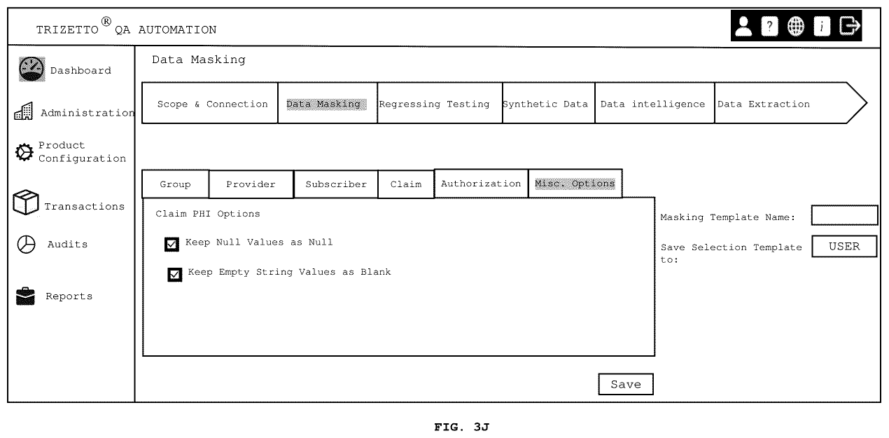

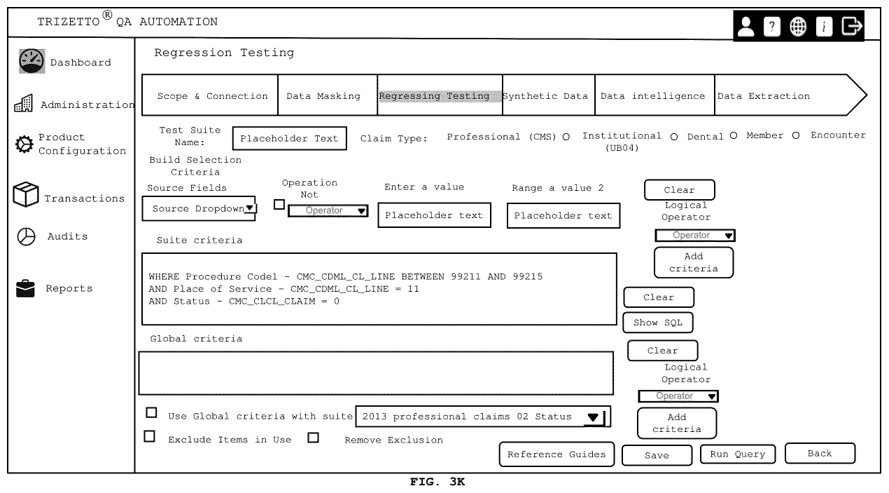

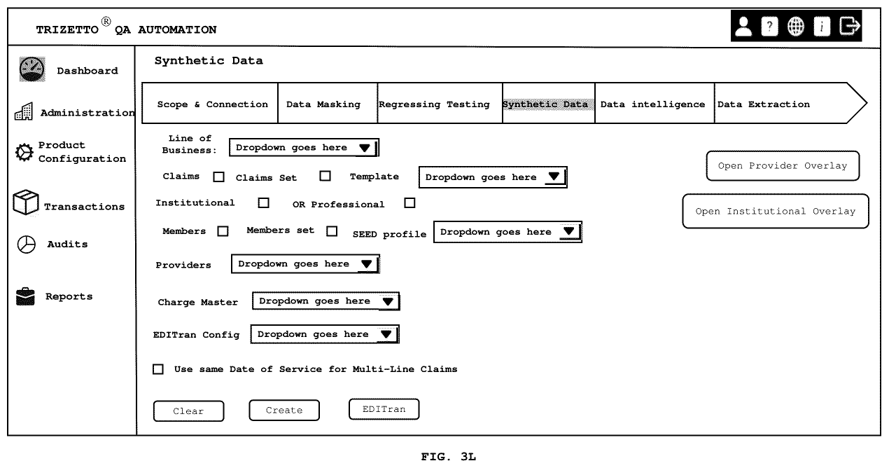

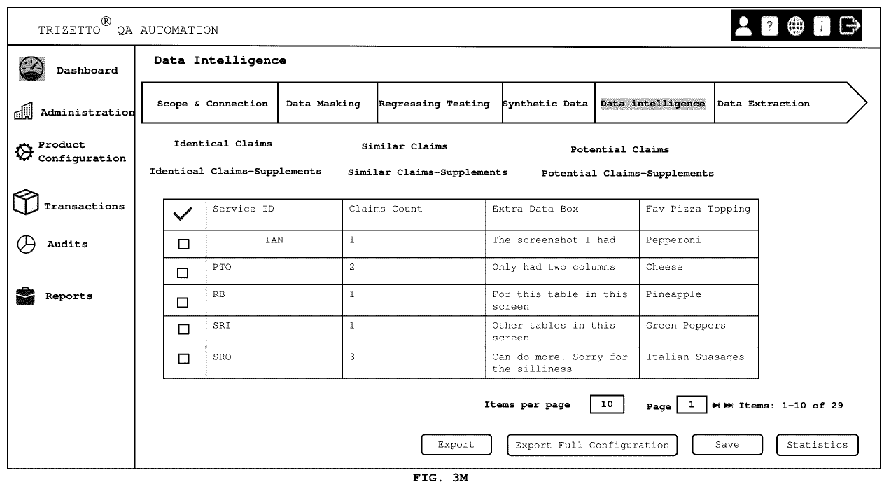

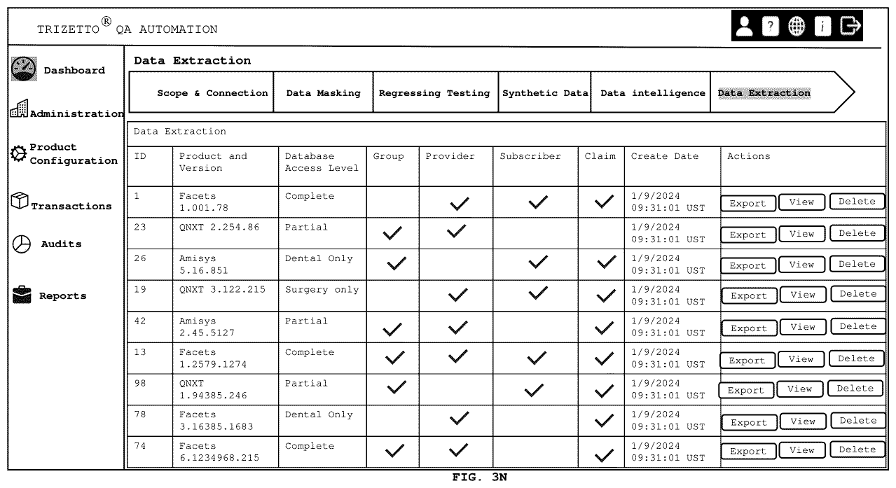

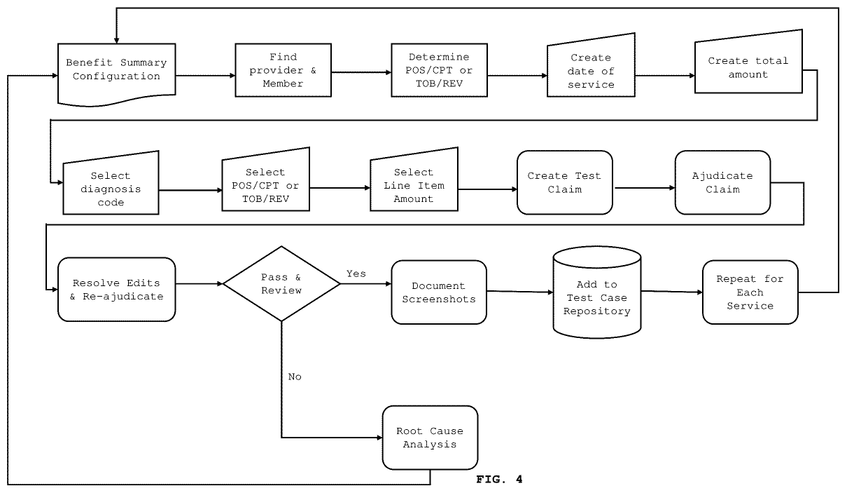

In an embodiment of the present invention, the test case execution unit 122 is configured to execute the generated test case by employing one or more automated execution techniques for testing the configuration data. The automated techniques used for test case execution include, but are not limited to, batch processing, robotic process automation (RPA), and technology services. The test cases are executed based on one or more test scope elements. In an embodiment of the present invention, the test case is executed against the test case data extracted from the test case data storage unit 126 for determining accuracy of the configuration data. FIGS. 3A-3N illustrate screenshots of a GUI depicting test case data extraction process for use in test case execution. In an example, the test scope elements relate to testing needs associated with claims such as edge test cases or test cases that either occur less frequently or are always adjudicated incorrectly. The test case execution unit 122 identifies such test cases based on a source environment and determines accuracy of the configuration data by obfuscating the PHI data and using the obfuscated PHI data with the synthetic healthcare claims datasets. The test case execution unit 122 is not required to re-work on the insurance claim data, penalty and payments for interest initial incorrect adjudication of the insurance claims. Further, the test case execution processes intermediary results prior to carrying out the next step of test case execution. In an exemplary scenario, all healthcare payers have data related to coverage benefit summaries or explanation of benefit coverage that provide covered and non-covered benefits which are configured for claims adjudication. The coverage benefit summaries include, but are not limited to, if benefit is covered or not covered based on insurance providers network status, if their id is deducible, co-insurance or co-payment owed by the patient and if there are any maximums present. The test case execution unit 122 generates the test cases for both positive testing and negative testing for testing the configuration data associated with benefits, as one benefit may have multiple configurations.

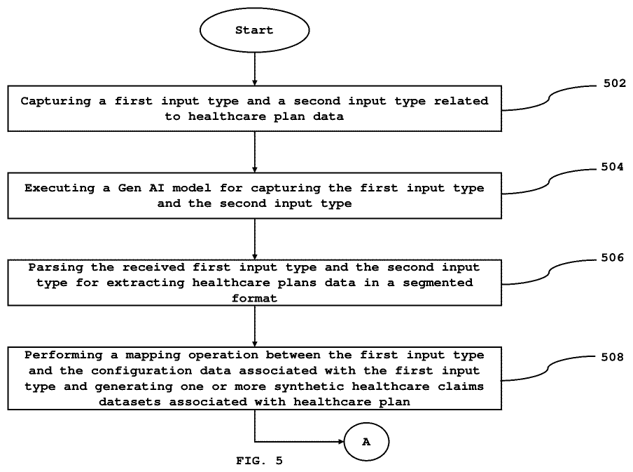

In an embodiment of the present invention, the test result evaluation and reporting unit 124 is configured to evaluate the execution of the test cases by comparing outcome of the test case execution with one or more pre-determined expected results. The pre-determined expected results comprise validation testing results or the regression or parallel testing results. In an embodiment of the present invention, the test result evaluation and reporting unit 124 executes an analysis model for reviewing and improving test case coverage with respect to a desired test cycle. For example, for some specific configuration common tests, the edge cases, or the negative cases are determined that would more comprehensively validate the configuration data. FIG. 4 illustrates a flowchart depicting the validation process of the configuration data. The evaluation results may be represented as, but not limited to, prior results, documented expectations, and confirmation of test case execution. The test case execution results may validate healthcare claims scope coverage and failures that may require retesting. In an embodiment of the present invention, the test result evaluation and reporting unit 124 is configured to render the test case execution results via the GUI on the input device 110 in the form of a report or logging of results along with specific recommendations of next steps. In another embodiment of the present invention, the test result evaluation and reporting unit 124 is configured to carry out data clean-up, data purging, data editing, data elimination or data staging that may be necessary based on the results of test case execution. In an embodiment of the present invention, the engine 104 is configured to provide data security during the Quality Assurance (QA) testing of configuration data related to healthcare claims. Data security is provided by implementing a Security Information and Event Management (SIEM) technique. The SIEM technique is implemented by integrating data processing tools including, but are not limited to, integrating Splunk® data with Elasticsearch®.

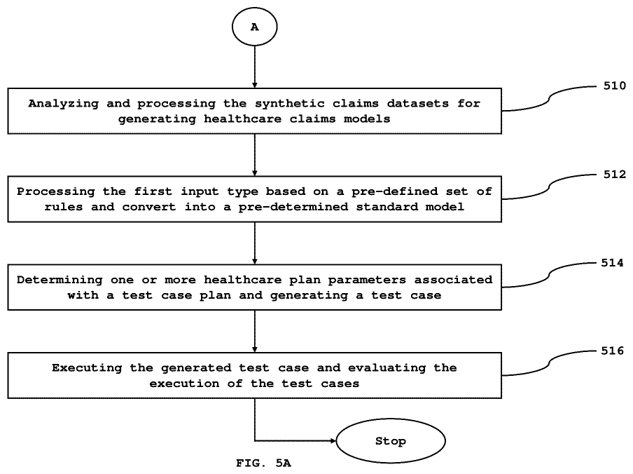

FIG. 5 and FIG. 5A illustrates a flowchart depicting a method for automated testing of configuration data, in accordance with an embodiment of the present invention.

At step 502, a first input type and a second input type related to healthcare plan data are captured. In an embodiment of the present invention, a Graphical User Interface (GUI) is rendered for providing dashboards in order to capture one or more inputs related to healthcare claims data. The GUI is developed using a distributed development and consolidated delivery approach. Further, the GUI is embedded with various web components for carrying out various functionalities of capturing healthcare claims data. In an embodiment of the present invention, the dashboard is configured to capture the first input type and the second input type related to healthcare plan data. In an exemplary embodiment of the present invention, the first input type relating to healthcare claims data may include, healthcare claims type (e.g., healthcare insurance plan), Evidence Of Healthcare Plan Coverage (EOC), healthcare benefit summaries, healthcare plan contract terms, documents related to healthcare plan, health issue coverage data related to healthcare plan, patient personal data, patient health issue data, previous patient data (e.g., prior year's health care claim may have been “cloned” and used to create basis for the new healthcare claims), healthcare claims data storage location. The first input type may be provided in a pre-defined manner including manually, by uploading directly or by fetching from the claims data storage unit 112 integrated with an input device 110. In an exemplary embodiment of the present invention, the second input type relating to healthcare claims testing data may include, but is not limited to, types of testing to be performed, and location of testing. The type of testing may include, but is not limited to, validation testing, and a regression or parallel testing.

At step 504, a Gen AI model is executed for capturing the first input type and the second input type. In an embodiment of the present invention, Gen AI model, and pre-generated AI and ML models are employed for processing the healthcare claims data based on the received first input type and the second input type. In an embodiment of the present invention, a Gen AI model such as Open AIR, Gemini®, Azure® open AI, etc., are executed to guide the user through the GUI for capturing the first input type and the second input type in an efficient manner. For example, the user may provide a prompt related to the QA testing of the configuration data for a healthcare plan. The prompt may include, but is not limited to, determining where the configuration data was generated, guidance on how the configuration should be tested, determining whether prior results exist, determining if similar configuration exists, determining scope of QA testing and what type of results are to be expected. In an embodiment of the present invention, a semantic similarity analysis is carried out for determining meaning of the prompt provided by the user. The words provided in prompts are converted to vectors. The words are converted to vectors by implementing techniques including, but are not limited to, a Global Vectors for Word Embeddings (GloVe) technique, auto-tokenizers, etc. Further, the vectors associated with semantically related words are clustered by determining proximity of the vectors. A K-means clustering technique is implemented for clustering the vectors associated with the semantically related words. In an embodiment of the present invention, the Gen AI model provides a required output to the user for suitably capturing the first input type and the second input type. In various embodiments of the present invention, an Artificial Intelligence (AI) based dictionary comprising of association of phrases, words and similies is created which is continuously updated and refined and carries out probabilistic prediction of the phrases, words and similies, thereby enhancing the AI-based dictionary's ability to mirror natural language use. Each time, for instance, healthcare plans data is processed a pre-trained database of word associations is provided in the form of the AI-based dictionary. The AI-based dictionary is a dynamic and intelligent RAG which is capable of evolving with language and providing valuable insights for various applications from natural language processing to content recommendation systems, and bridging the gap between human language and machine understanding.

At step 506, the received first input type and the second input type are parsed for extracting healthcare plans data in a segmented format. In an embodiment of the present invention, subsequent to parsing, a configuration data document analysis model is executed for interpreting the first input type based on evaluating and identifying one or more relationships between the different type of healthcare claims data associated with the first input type. The interpretation of the first input type is carried out by identifying one or more characteristics associated with the first input type and the second input type. The identified characteristics include, but are not limited to, determining type of health issue associated with a particular patient (e.g., broken arm is common with people of 12 years of age, if during a dermatologist visit a spot is noted, then it might be melanoma, etc.), explanation of healthcare claims coverage, healthcare claims benefit summaries, healthcare claims configuration document types, determining claims testing scope (e.g., determining claims testing needs such as positive/negative testing which applications of functions need to be included, etc.), determining form of claims test result confirmation, etc.

At step 508, a mapping operation is performed between the first input type and the configuration data associated with the first input type and one or more synthetic healthcare claims datasets associated with healthcare plan are generated. In an embodiment of the present invention, the mapping operation is performed between the first input type and the configuration data associated with the first input type based on the interpretation. For example, if the first input type relates to a claims benefit summary and is presented in a natural language form, then the first input type is mapped to the configuration data relating to Current Procedural Terminology (CPT) codes that are encoded in the configuration of a particular healthcare claims processing tool. In an embodiment of the present invention, one or more synthetic healthcare claims datasets associated with healthcare plan are generated by executing a regression or classification machine learning model. The synthetic dataset claims are used for processing the healthcare claims. In an embodiment of the present invention, the synthetic healthcare claims datasets are generated based on the identified relationships between the different type of first input type, the characteristics associated with the first input type, and the second input type. In an example, for a particular healthcare claim, the “best, less common, or rare” characteristics for patient age, patient gender, physician type, facility type, cost share, etc., may be identified. The synthetic claims datasets are similar to actual Personal Health Information (PHI) of a patient. Advantageously, by employing synthetic claims datasets, privacy concerns associated with the usage of PHI are eliminated. The generated synthetic claims datasets also include information on a type of testing which is required to be carried out for the healthcare claims data. In an exemplary embodiment of the present invention, the type of testing includes regression or parallel testing and validation testing.

At step 510, the synthetic claims datasets are analyzed and processed for generating healthcare claims models. In an embodiment of the present invention, large dataset analysis and processing techniques are employed along with LLM management techniques for analyzing and processing the synthetic claims datasets. The large dataset analysis and processing techniques include, but are not limited to, Python® libraries like NumPy®, Pandas®, Shap® and Sklearn®. The LLM management techniques include, but are not limited to, Azure® ML and SQL Server. In an embodiment of the present invention, a configuration data analysis technique is executed to generate the healthcare claim models.

In an embodiment of the present invention, one or more components associated with the synthetic claims datasets are evaluated for healthcare claims processing and a suitable methodology for healthcare claims data processing is determined. In an embodiment of the present invention, the prompts related to user interactions are included to validate the analysis process and identify characteristics associated with the first input type. For example, the prompts relate to best source for test case, scope of healthcare claims testing, mappings carried out, and one or more functional elements to be included in the build of the test case.

At step 512, the first input type is processed based on a pre-defined set of rules and converted into a pre-determined standard model. In an embodiment of the present invention, the pre-defined set of rules executes an Application Programming Interface (API) for extracting a first set of rules from the claims data storage unit 112. The fetched first input type is loaded in a high structure SQL Server database based on a set of pre-defined configurations. The extraction process creates an event bus message using a queuing protocol (e.g., a Rabbit MQR) which fetches the first input type data from the SQL Server database or from one or more third-party sources and converts the first input type into a pre-determined standard model including a NoSQL database schema standard (e.g., Fast Healthcare Interoperability Resources (FHIR) R5 standard). The pre-defined standard model is saved into a low structure database (e.g., MongoDB®) (not shown) with a unique data set ID. The pre-defined standard model is referred to as a data puddle which is in a pre-defined file format (e.g., as FHIR JSON). The data puddle is a discrete set of data. In an embodiment of the present invention, the first input type is masked prior to converting into the pre-defined standard model by executing a set of data alteration rules with respect to a database schema.

In an embodiment of the present invention, the pre-defined standard model stored in the low structure database is further retrieved and an event is triggered for formatting the pre-defined standard model. In an exemplary embodiment of the present invention, a data formatting application (e.g., EDITran) is executed for formatting the pre-defined standard model. After completion of the formatting event, a data file in a pre-defined file format is generated. For example, an X12 file, a JSON, a flat file is generated in a specified output folder. In an embodiment of the present invention, the formatted pre-defined standard model is transmitted to a target database using a batch process upload technique for storage, re-use and retrieval of the test case data. In an embodiment of the present invention, a backup of the data puddles is created by using a database tool to create a full backup of the collections and a purging process is executed weekly for carrying out scans through the collection of data puddles older than the number of days set as retention days in a configuration file.

At step 514, one or more healthcare plan parameters associated with a test case plan are determined and a test case is generated. In an embodiment of the present invention, firstly, one or more healthcare plan parameters associated with a test case plan are determined. The one or more healthcare plan parameters, include, but are not limited to, determining specific service and diagnosis codes to match the requirements and descriptions of the configuration, healthcare claims, rules, billing, enrollment, contracts, authorizations, and identifying supporting particulars relating to implementing various encoded configuration data. Further, a test case is generated by carrying out search and cloning of the one or more healthcare plan parameters associated with the test case plan. In an exemplary embodiment of the present invention, the configuration data analysis technique and data cloning and PHI obfuscation are employed for generating the test case. In an example, PHI data may be used for testing. By obfuscating the PHI data for generating the test case it becomes difficult to find the obfuscated PHI data and, therefore, it is not possible to unmask the obfuscated PHI data, thereby providing security. In an embodiment of the present invention, the one or more healthcare plan parameters are synthetically generated by using synthetic data creation technique. In an embodiment of the present invention, test case generation also includes generating data relating to proper execution of the test case, such as authorization, referrals, or care components. Further, the synthetic healthcare claims datasets are fetched for embedding with the generated test case.

At step 516, the generated test case is executed and the execution of the test cases is evaluated. In an embodiment of the present invention, the generated test case is executed by employing one or more automated execution techniques for testing the configuration data. The automated techniques used for test case execution include, but are not limited to, batch processing, robotic process automation (RPA), and technology services. The test cases are executed based on one or more test scope elements. In an embodiment of the present invention, the test case is executed against the test case data extracted from for determining accuracy of the configuration data. In an example, the test scope elements relate to testing needs associated with claims such as edge test cases or test cases that either occur less frequently or are always adjudicated incorrectly. Such test cases are identified based on a source environment and accuracy of the configuration data is determined by obfuscating the PHI data and using t the obfuscated PHI data with the synthetic healthcare claims datasets. Further, the test case execution processes intermediary results prior to carrying out the next step of test case execution.

In an embodiment of the present invention, the execution of the test cases is evaluated by comparing outcome of the test case execution with one or more pre-determined expected results. The pre-determined expected results comprise validation testing results or regression or parallel testing results. In an embodiment of the present invention, an analysis model is executed for reviewing and improving test case coverage with respect to a desired test cycle. For example, for some specific configuration common tests, the edge cases, or the negative cases are determined that would more comprehensively validate the configuration data. The evaluation results may be represented as, but not limited to, prior results, documented expectations, and confirmation of test case execution. The test case execution results may validate healthcare claims scope coverage and failures that may require retesting. In an embodiment of the present invention, the test case execution results are rendered via the GUI in the form of a report or logging of results along with specific recommendations of next steps. In another embodiment of the present invention, data clean-up, data purging, data editing, data elimination or data staging are carried out that may be necessary based on the results of test case execution.

Advantageously, in accordance with various embodiments of the present invention, the present invention provides for efficient automated testing of configuration data by employing AI and ML techniques and Gen AI techniques in real-time. The present invention provides for streamlining complex tasks across various domains. The present invention focuses on automation of translating working files/formats into structured formats, which is crucial for data analysis and system integration. Also, the present invention provides for creating a mapping document that defines rules for transformation, thereby ensuring consistency and accuracy in converting data. The present invention further provides for enhancing software testing efficiency by automating generation of test cases based on specific coverage information. This reduces manual effort required in testing and improves reliability of the testing process. Further, the present invention provides for effectively addressing concerns of data protection and privacy associated with configuration data testing by using synthetic datasets and masking inputs. The present invention provides for rendering an intelligent GUI for easy testing of configuration data associated with the healthcare claims. Further, the present invention provides for carrying out accurate, adequate, time efficient and cost-effective QA testing of configuration data. Yet further, the present invention provides for leveraging advanced machine models learning for more accurate understanding of claim descriptions, facilitating more efficient and accurate processing of healthcare claims and prediction of healthcare claim outcomes, preventing errors through real-time checks patient records, and medical guidelines detecting potential fraud, and determining reimbursement amount with greater accuracy. The present invention models progression of medical procedures, represented by, for example, CPT codes and values and Systematized Nomenclature of Medicine (SNOMED) clinical terms, within a patient group, which is particularly beneficial for predicting healthcare trends and preparing for patient care requirements. Further, the present invention provides for continuous learning and adaptation from historical data and user interactions, thereby providing fast healthcare claims processing. Yet further, the present invention provides a robust framework for identifying data anomalies, enhancing predictive analysis, optimizing workflows, and ensuring data integrity and high-quality outcomes across various industries.

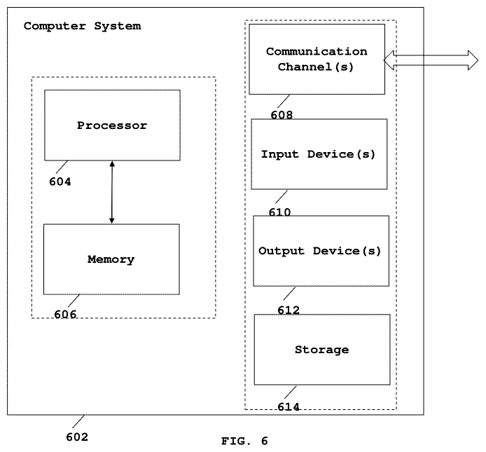

FIG. 6 illustrates an exemplary computer system in which various embodiments of the present invention may be implemented. The computer system 602 comprises a processor 604 and a memory 606. The processor 604 executes program instructions and is a real processor. The computer system 602 is not intended to suggest any limitation as to scope of use or functionality of described embodiments. For example, the computer system 602 may include, but not limited to, a programmed microprocessor, a micro-controller, a peripheral integrated circuit element, and other devices or arrangements of devices that are capable of implementing the steps that constitute the method of the present invention. In an embodiment of the present invention, the memory 606 may store software for implementing various embodiments of the present invention. The computer system 602 may have additional components. For example, the computer system 602 includes one or more communication channels 608, one or more input devices 610, one or more output devices 612, and storage 614. An interconnection mechanism (not shown) such as a bus, controller, or network, interconnects the components of the computer system 602. In various embodiments of the present invention, operating system software (not shown) provides an operating environment for various softwares executing in the computer system 602 and manages different functionalities of the components of the computer system 602.

The communication channel(s) 608 allows communication over a communication medium to various other computing entities. The communication medium provides information such as program instructions, or other data in a communication media. The communication media includes, but are not limited to, wired or wireless methodologies implemented with an electrical, optical, RE, infrared, acoustic, microwave, Bluetooth, or other transmission media.

The input device(s) 610 may include, but not limited to, a keyboard, mouse, pen, joystick, trackball, Brain Computer Interface (BCI), a scanning device, touch screen or any another device that is capable of providing input to the computer system 602. In an embodiment of the present invention, the input device(s) 610 may be a sound card or similar device that accepts audio input in analog or digital form. The output device(s) 612 may include, but not limited to, a user interface on CRT or LCD, printer, speaker, CD/DVD writer, or any other device that provides output from the computer system 602.

The storage 614 may include, but not limited to, magnetic disks, magnetic tapes, CD-ROMs, CD-RWs, DVDs, flash drives or any other medium which can be used to store information and can be accessed by the computer system 602. In various embodiments of the present invention, the storage 614 contains program instructions for implementing the described embodiments.

The present invention may suitably be embodied as a computer program product for use with the computer system 602. The method described herein is typically implemented as a computer program product, comprising a set of program instructions which is executed by the computer system 602 or any other similar device. The set of program instructions may be a series of computer readable codes stored on a tangible medium, such as a computer readable storage medium (storage 614), for example, diskette, CD-ROM, ROM, flash drives or hard disk, or transmittable to the computer system 602, via a modem or other interface device, over either a tangible medium, including but not limited to optical or analogue communications channel(s) 608. The implementation of the invention as a computer program product may be in an intangible form using wireless techniques, including but not limited to microwave, infrared, Bluetooth, or other transmission techniques. These instructions can be preloaded into a system or recorded on a storage medium such as a CD-ROM or made available for downloading over a network such as the internet or a mobile telephone network. The series of computer readable instructions may embody all or part of the functionality previously described herein.

The present invention may be implemented in numerous ways including as a system, a method, or a computer program product such as a computer readable storage medium or a computer network wherein programming instructions are communicated from a remote location.

While the exemplary embodiments of the present invention are described and illustrated herein, it will be appreciated that they are merely illustrative. It will be understood by those skilled in the art that various modifications in form and detail may be made therein without departing from or offending the scope of the invention.

We claim:

1. A system for automated testing of configuration data, the system comprising:
a memory storing program instructions;
a processor executing instructions stored in the memory; and
a data testing engine executed by the processor and configured to:
capture a first input type and a second input type related to healthcare claims data via a Graphical User Interface (GUI) by executing a Generative Artificial Intelligence (Gen AI) model;
parse the first input type and the second input type for extracting healthcare plans data in a segmented format, wherein a configuration data document analysis model is executed for determining relationships between different types of the healthcare claims data associated with the first input type and identifying one or more characteristics associated with the first input type;
generate synthetic healthcare claims datasets based on the determined relationships and identified characteristics associated with the first input type and the second input type;
generate a test case by carrying out search and cloning of one or more healthcare plan parameters associated with a test case plan, wherein the synthetic healthcare claims datasets are embedded into the generated test case; and
execute the generated test case based on one or more test scope elements for testing the configuration data and determining accuracy of the configuration data.

2. The system as claimed in claim 1, wherein the first input type relates to healthcare claims data comprising healthcare claims type, Evidence Of Healthcare Plan Coverage (EOC), healthcare benefit summaries, healthcare plan contract terms, documents related to healthcare plan, health issue coverage data related to healthcare plan, patient personal data, patient health issue data, previous patient data, and healthcare claims data storage location, and wherein the second input type relates to healthcare claims testing data comprising types of testing to be performed, and location of testing, and wherein the type of testing comprises a validation testing and a regression or parallel testing.

3. The system as claimed in claim 1, wherein the data testing engine comprises an automation unit executed by the processor and configured to communicate with a UI rendering and input capturing unit to execute the Gen AI model to guide a user through the GUI for capturing the first input type and the second input type efficiently, and execute the Gen AI model and pre-generated AI and ML models for processing the healthcare claims data based on the first input type and the second input type.

4. The system as claimed in claim 3, wherein a semantic similarity analysis is carried out by the automation unit for determining meaning of a prompt provided by the user related to capturing the first input type and the second input type, and wherein the automation unit is configured to convert the words provided in prompts to vectors, and wherein the vectors associated with semantically related words are clustered by the automation unit by determining proximity of the vectors.

5. The system as claimed in claim 1, wherein the automation unit implementing the Gen AI model provides a required output for suitably capturing the first input type and the second input type, and wherein the automation unit creates an Artificial Intelligence (AI) based dictionary comprising of association of phrases, words and similies which is continuously updated and refined and carries out probabilistic prediction of the phrases, words and similies, and wherein each time healthcare plans data is processed the automation unit communicates to and provides for a pre-trained database of word associations in the form of the AI-based dictionary.

6. The system as claimed in claim 1, wherein the data testing engine comprises an automation unit executed by the processor and is configured to perform a mapping operation between the first input type and a configuration data associated with the first input type, and wherein the automation unit generates the one or more synthetic healthcare claims datasets by executing a regression or classification machine learning model, and wherein the synthetic dataset claims are used for processing the healthcare claims, and wherein the generated synthetic claims datasets comprises information on a type of testing which is required to be carried out for the healthcare claims data.

7. The system as claimed in claim 1, wherein the data testing engine comprises a data analysis, acquisition and loading unit executed by the processor and is configured to analyze and process the synthetic claims datasets for generating healthcare claims models, and wherein large dataset analysis and processing techniques are employed along with LLM management techniques for analyzing and processing the synthetic claims datasets, and wherein the data analysis, acquisition and loading unit executes a configuration data analysis technique to generate the healthcare claim models.

8. The system as claimed in claim 1, wherein the data testing engine comprises a data analysis, acquisition and loading unit executed by the processor and is configured to evaluate one or more components associated with the synthetic claims datasets for healthcare claims processing and determine a suitable methodology for healthcare claims data processing, and wherein prompts related to user interactions are included to validate the analysis process and identified characteristics with the first data type.

9. The system as claimed in claim 1, wherein the data testing engine comprises a data analysis, acquisition and loading unit executed by the processor and is configured to extract and process the first input type fetched from a claims data storage unit based on a pre-defined set of rules and convert the first input type into a pre-determined standard model, and wherein the pre-defined set of rules executes an Application Programming Interface (API) for extracting a first set of rules from the claims data storage unit.

10. The system as claimed in claim 9, wherein the fetched first input type is loaded in a high structure SQL Server database based on a set of pre-defined configurations, and wherein the extraction process creates an event bus message using a queuing protocol which fetches the first input type data from an SQL Server database or from one or more third-party sources and converts the first input type into the pre-determined standard model including a NoSQL database schema standard.

11. The system as claimed in claim 10, wherein the pre-defined standard model is saved into a low structure database with a unique data set ID, and wherein the pre-defined standard model is a data puddle which is in a pre-defined file format, and wherein the first input type is masked prior to converting into the pre-defined standard model by executing a set of data alteration rules with respect to a database schema.

12. The system as claimed in claim 11, wherein the pre-defined standard model stored in the low structure database is retrieved and an event is triggered for formatting the pre-defined standard model, and after completion of the formatting event a data file in a pre-defined file format is generated, and wherein the formatted pre-defined standard model is transmitted to a test case data storage unit which is a target database using a batch process upload technique for storage, re-use and retrieval of the test case data.

13. The system as claimed in claim 1, wherein the data testing engine comprises a test case generation unit executed by the processor and is configured to determine the one or more healthcare plan parameters, comprising determining specific service and diagnosis codes to match the requirements and descriptions of the configuration, healthcare claims, rules, billing, enrollment, contracts, authorizations, and identifying supporting particulars relating to implementing various encoded configuration data, and wherein the one or more healthcare plan parameters are synthetically generated by using synthetic data creation technique.

14. The system as claimed in claim 1, wherein a configuration data analysis technique and a data cloning and Personal Health Information (PHI) obfuscation are employed for generating the test case.

15. The system as claimed in claim 1, wherein the data testing engine comprises a test case execution unit executed by the processor and is configured to execute the test case by employing one or more automated execution techniques comprising batch processing, robotic process automation (RPA), and technology services, and wherein the test case is executed against the test case data extracted from a test case data storage unit for determining accuracy of the configuration data, and wherein the test case execution processes intermediary results prior to carrying out the next step of test case execution.

16. The system as claimed in claim 1, wherein the data testing engine comprises a test result evaluation and reporting unit executed by the processor and is configured to evaluate execution of the test case by comparing outcome of the test case execution with one or more pre-determined expected results, and wherein the pre-determined expected results comprises validation testing results or regression or parallel testing results, and wherein the test result evaluation and reporting unit executes an analysis model for reviewing and improving test case coverage with respect to a desired test cycle.

17. The system as claimed in claim 17, wherein the evaluation results comprise prior results, documented expectations, and confirmation of test case execution, and wherein the test case execution results validate healthcare claims scope coverage and failures that require retesting, and wherein the test case execution results are rendered via the GUI on an input device in the form of a report or logging of results along with specific recommendations of next steps.

18. A method for automated testing of configuration data, the method is implemented by a processor executing instructions stored in a memory, the method comprises:
capturing a first input type and a second input type related to healthcare claims data via a Graphical User Interface (GUI) by executing a Generative Artificial Intelligence (Gen AI) model;
parsing the first input type and the second input type for extracting healthcare plans data in a segmented format, wherein a configuration data document analysis model is executed for determining one or more relationships between different types of the healthcare claims data associated with the first input type and identifying one or more characteristics associated with the first input type;
generating synthetic healthcare claims datasets based on the determined relationships and identified characteristics associated with the first input type and the second input type;
generating a test case by carrying out search and cloning of one or more healthcare plan parameters associated with a test case plan, wherein the synthetic healthcare claims datasets are embedded into the generated test case; and
executing the generated test case based on one or more test scope elements for testing the configuration data and determining accuracy of the configuration data.

19. The method as claimed in claim 18, wherein the step of capturing comprises carrying out a semantic similarity analysis for determining meaning of a prompt provided by a user, and wherein the words provided in prompts are converted to vectors, and wherein the vectors associated with semantically related words are clustered by determining proximity of the vectors.

20. The method as claimed in claim 18, wherein the step of capturing comprises providing by the Gen AI model a required output for suitably capturing the first input type and the second input type, and wherein an Artificial Intelligence (AI) based dictionary is created comprising of association of phrases, words and similies which is continuously updated and refined and probabilistic prediction of the phrases, words and similies is carried out, and wherein a pre-trained database of word associations is provided in the form of the AI-based dictionary each time the healthcare plans data is processed.

21. The method as claimed in claim 19, wherein the step of parsing comprises performing a mapping operation between the first input type and the configuration data associated with the first input type.

22. The method as claimed in claim 18, wherein the step of generating synthetic data comprises executing a regression or classification machine learning model, and wherein the synthetic dataset claims are used for processing the healthcare claims, and wherein the generated synthetic claims datasets comprise information on a type of testing which is required to be carried out for the healthcare claims data.

23. The method as claimed in claim 22, wherein the synthetic claims datasets are analyzed and processed for generating healthcare claims models, and wherein large dataset analysis and processing techniques are employed along with LLM management techniques for analyzing and processing the synthetic claims datasets, and wherein a configuration data analysis technique is executed to generate the healthcare claim models.

24. The method as claimed in claim 1, wherein the method comprising fetching the first input type from a claims data storage unit and processing the first input type based on a pre-defined set of rules; and
converting the first input type into a pre-determined standard model, wherein the pre-defined set of rules executes an Application Programming Interface (API) for extracting a first set of rules from the claims data storage unit.

25. The method as claimed in claim 24, wherein the fetched first input type is loaded in a high structure SQL Server database based on a set of pre-defined configurations, and wherein the extraction process creates an event bus message using a queuing protocol which fetches the first input type data from an SQL Server database or from one or more third-party sources and converts the first input type into the pre-determined standard model including a NoSQL database schema standard, and wherein the pre-defined standard model is saved into a low structure database with a unique data set ID, and wherein the pre-defined standard model is a data puddle which is in a pre-defined file format, and wherein the first input type is masked prior to converting into the pre-defined standard model by executing a set of data alteration rules with respect to a database schema.

26. The method as claimed in claim 18, wherein the step of execution of the test case comprises evaluating the test cases by comparing outcome of the test case execution with one or more pre-determined expected results, and wherein the pre-determined expected results comprises validation testing results or the regression or parallel testing results, and wherein an analysis model is executed for reviewing and improving test case coverage with respect to a desired test cycle.

27. A computer program product comprising:
a non-transitory computer-readable medium having computer program code stored thereon, the computer-readable program code comprising instructions that, when executed by a processor, causes the processor to:
capture a first input type and a second input type related to healthcare claims data via a Graphical User Interface (GUI) by executing a Generative Artificial Intelligence (Gen AI) model;
parse the first input type and the second input type for extracting healthcare plans data in a segmented format, wherein a configuration data document analysis model is executed for determining relationships between different types of the healthcare claims data associated with the first input type and identifying one or more characteristics associated with the first input type;
generate synthetic healthcare claims datasets based on the determined relationships and the identified characteristics associated with the first input type and the second input type;
generate a test case by carrying out search and cloning of one or more healthcare plan parameters associated with a test case plan, wherein the synthetic healthcare claims datasets are embedded into the generated test case; and
execute the generated test case based on one or more test scope elements for testing the configuration data and determining accuracy of configuration data.
